(19) United States
(12) Patent Application Publication
(10) Pub. No.: US 2006/0257977 A1
(43) Pub. Date: Nov. 16, 2006

(54) Slowly digestible starch

(76) Inventors: Bruce R. Hamaker, West Lafayette, IN (US); Xian-Zhong Han, West Lafayette, IN (US)

Correspondence Address: GREENLEE WINNER AND SULLIVAN P C, 4875 PEARL EAST CIRCLE, SUITE 200, BOULDER, CO, 80301, US

(21) Appl. No.: 10/543,705

(22) PCT Filed: Jan. 28, 2004
(86) PCT No.: PCT/US04/02566

§ 371 Date: Jun. 1, 2006

Related U.S. Application Data
(60) Provisional application No. 60/443,229, filed on Jan. 28, 2003.

(51) Int. Cl. C12P 19/20

(52) U.S. Cl. 435/96

(57) ABSTRACT

The invention provides processes to make slowly digestible starches from native and commercial starches. Slowly digestible starches are prepared by controlled hydrolysis of gelatinized starch by alpha amylase. The slowly digestible starches have a range of starch digestion rates and fall between normal, untreated commercial and native starch, and commercial resistant starches. The slowly digestible starches provide a range of starch functionalities. These slow digesting starches retain their digestion characteristic after cooling, and can used in a range of processed food products to modulate the rapid glucose release typical of many processed starchy foods. Edible products incorporating slowly digestible starch will exhibit lower glycemic index and increase satiety. The invention provides solid and liquid food, nutritional, and drug preparations containing the slowly digestible starch. The invention further provided edible products for extended energy release for example, for use in sports drinks and snack bars. The slowly digestible starches can also be employed as functional food grade additives to provided beneficial rheological or other properties to edible compositions.

### BACKGROUND OF THE INVENTION

The present invention relates to a method of making slowly digestible starch generally useful in food preparation and manufacture.

Historically, the issue of whether starches in foods are digested quickly or slowly has been given little attention, except for diabetic patients who are typically prescribed diet changes to balance blood glucose levels. Accordingly the USDA Diet Pyramid formulated in the early 1980's providing dietary guidelines for the U.S. population did not discriminate between fast and slow digesting starches in matters of personal health. The Diet Pyramid helped to generate recognition among consumers and food processors that a diet high in carbohydrates and low in fat was desirable for alleviating a number of chronic health problems, including obesity and cardiovascular diseases. These diet recommendations was embraced by a majority of the public, although opposing and strongly held viewpoints arose purporting that high protein/fat and low carbohydrate diets also address these same ailments. In spite of the acceptance the high carbohydrate/low fat hypothesis and introduction of a multitude of food products based on and the diet regimes following it, the incidence of obesity has increased dramatically over the last two decades. Energy intake per adult has increased about 400 calories/day. Incidence of diabetes in the same period has likewise risen at a rate higher than any other disease. In response to this troubling picture, investigators and policymakers are taking another look at dietary recommendations, including an examination of starch digestion rate and its effect on insulin response, satiety, and a range of other physiological responses. There is compelling evidence that slowly digesting (or slow glucose release), as well as other low glycemic index, starches give a satiety effect and moderate blood glucose and insulin levels that may act to combat the onset of diabetes and obesity-related disorders.

The starch industry has up to this point focused chiefly on ways to create resistant starches that are not digested by host enzymes. Commercial resistant starches presently available include Novalose™ products (National Starch Co.), ActiStar™ products (Cargill/Cerestar), and CrystaLean™ products (Opta Foods, Bedford Mass.). No commercial product of slowly digestible starch is currently available on the market. A number of patents describe methods for making resistant starches as well as properties and applications of such starches. See, for example, U.S. Pat. Nos. 6,664,389; 6,623,943; 6,090,594; 6,043,229; 5,962,047; 5,855,946; 5,849,090; 5,051,271; and 3,729,380, U.S. published application 2003/0094172 (published May 22, 2003), and EP applications 688,872 and 564,893.

U.S. Pat. No. 5,612,202 relates to non-random cleavage of starch using Bacillus stereothermophilus alpha-amylase in a process to ultimately produce maltodextrin having a D.E. of less than about 8. U.S. Pat. No. 5,599,569 relates to the use of amylase treated low-viscosity starch in foods, for example to adhere seasoning to a food product. U.S. Pat. No. 5,562,937 relates to an amylase-treated waxy starch for use in foods.

U.S. applications 2003/0219520 (published Nov. 27, 2003) and 2003/0215562 (published Nov. 20, 2003) relate to slowly digestible starch products. The applications relate to debranching of amylose-containing starches and low-amylose starches employing isoamylase and pullulanase which hydrolyze the 1, 6-alpha-D-glucosidic linkages of glucan polymers, such as amylopectin. The slowly digestible starch compositions are at least about 90% debranched. These published applications are incorporated by reference herein in their entirety.

### SUMMARY OF THE INVENTION

The present invention relates to starch products which are modified by controlled digestion with alpha-amylase. The modified starches of this invention are partially crystalline starches, containing some branched glucans and shorter chain amylaose, that on consumption by an individual are digested more slowly, or to a lesser degree, than untreated gelatinized starches (e.g., native starches). However, in contrast to resistant starches, the slowly digestible starches of this invention are substantially digested (about 80% or more and preferably more than 90%) in the small intestine.

This invention provides slowly digestible starch compositions prepared from untreated starches (e.g., starches extracted from plants) by controlled digestion with alpha-amylase. The slowly digestible starch compositions are non-granular, at least partially crystalline starch, that on consumption by an individual, are digested more slowly than the native starch from which it is made. Slowly digestible starch compositions comprise branched glucans which are believed to, at least in part, provide slow digestibility. Slowly digestible starch compositions can further comprise intermediate length linear glucans (amylose), particularly those ranging in molecular weight from about 1000,000 to about 10,000 D, or having DP ranging from about 50 to aobut 500. Unbranched glucans may also contribute to the slow digestibility of the starches of this invention.

The invention provides starch compositions which comprise at least about 30% to about 80% by weight of slowly digestible starch comprising branched glucans. The invention provides starch compositions which comprise at least about 50% to about 80% by weight of slowly digestible starch comprising branched glucans. Slowly digestible starch of this invention can also comprise intermediate length linear glucans. The alpha-amylase treated starches of this invention can include minor amounts of resistant starches, i.e., up to about 20% by weight resistant starch. The invention provides slowly digestible starch comprising branched glucans which contain a higher percentage of high molecular weight branches (those branches having DP of 25-100 or more) compared to the starch from which the slowly digestible starch is obtained by alpha-amylase treatment. The branched glucans of the slowly digestible starches of this invention are believed to result from preferential hydrolysis of the shorter branches of amylopectin in the starch starting material. Intermediate length linear glucans (amylose) are also believed to result from digestion of at least partially retrograded amylose. The branched glucans alone or in combination with the intermediate length linear glucans are believed responsible for the slowly digestible property of the treated starch.

In an embodiment, the invention provides slowly digestible starch which comprises less than about 50% by weight amylose. In another embodiment, the invention provides slowly digestible starch which comprises less than about 25% by weight amylose. In another embodiment, the invention provides slowly digestible starch which comprises 25% or more by weight of branched glucans. In another embodiment, the inveniton provides a slowly digestible starch which comprises 50% or more by weight of branched glucans. In an embodiment, the invention provides slowly digestible starch which comprises less than about 25% by weight amylose and more than about 75% by weight of branched glucans. In an embodiment, the invention provides slowly digestible starch which comprises less than about 50% by weight amylose and more than about 50% by weight of branched glucans.

In specific embodiments, the invention provides slowly digestible starch which has a D.E. of about 2 or less or a D.E. of about 1 or less.

The invention provides methods for preparing slowly digestible starch compositions in which gelatinized native starch is subject to controlled alpha-amylase digestion to preferentially remove short chain branches, e.g., branches having DP ranging from about 8 to about 25, from amylopectin of native starch. The alpha-amylase heated starch compositions comprise branched glucans derived from partial hydrolysis of amylopectin. Alpha-amylase digestion of at least partially retrograded or crystalline amylase is believed to result in intermediate length linear glucans (e.g., having DP between about 50 and about 500). The branched glucans alone or in combination with the intermediate length linear glucans is believed responsible for the slow digestibility of the treated starches of this invention.

The invention provides methods for preparing slowly digestible starch compositions in which gelatinized starch is allowed to at least partially retrograde prior to controlled alpha-amylase digestion to selectively remove shorter chain branches of amylopectin of the native starch and shorten amylose chains. The alpha-amylase treated starch compositions are at least partially crystalline and comprise branched glucans from amylopectin digestion and can further contain intermediate length amylose chains.

In an embodiment of the invention, gelatinized starch is subjected to controlled alpha-amylase digestion such that branched glucan is made having different branching structure and distribution of branch lengths compared to that of the amylopectin of the starch prior to treatment. In an embodiment of the invention, gelatinized starch is subjected to controlled alpha-amylase digestion such that amylose in the starch starting material is reduced to intermediate chain lengths ranging from a DP of about 50 to about 500.

In an embodiment of the invention, gelatinized starch is subjected to controlled alpha-amylase digestion such that the branched glucan of the treated starch contains a higher amount of longer chain, e.g., higher DP branches than the amylopectin of the native starch. More specifically the gelatinized starch is subjected to controlled alpha-amylase digestion such that the branched glucan of the treated starch contains a higher percentage of branches having DP higher than about 25 to about 100 than the amylopectin of the starch prior to treatment. More specifically the gelatinized starch is subjected to controlled alpha-amylase digestion such that the branched glucan of the treated starch contains a higher percentage of branches having DP higher than 65 to 100 than the amylopectin of the starch before treatment.

In a specific embodiment of the invention, gelatinized starch is subjected to controlled alpha-amylase digestion such that the ratios of two or more peaks in the branching distribution of the alpha-amylase treated starch is within a desired range. For example, for alpha-amylase treated corn starch digestion is continued until the ratio of the DP13/DP 32 peaks is less than that obtained in an analogous analysis of the starting untreated corn starch (see Example 4, below). A decrease in the ratio of DP13/DP32 of between about 30-70% provides slowly digestible starch products.

In an embodiment of the invention, gelatinized starch is subjected to controlled alpha-amylase digestion such that the molecular weight of the treated starch ranges from between about 10⁷ D to about 10⁴ D. In another embodiment of the invention, gelatinized starch is subjected to controlled alpha-amylase digestion such that the molecular weight of the treated starch ranges from between about 10⁶ D to about 10⁴ D. In another embodiment of the invention, gelatinized starch is subjected to controlled alpha-amylase digestion such that the molecular weight of the treated starch ranges from between about 10⁵ D to about 10⁴ D.

In an embodiment of the invention, gelatinized starch is subjected to controlled alpha-amylase digestion such that a branched glucan having a molecular weight between about 10⁷ D to about 10⁴ D is formed. In another embodiment, gelatinized starch is subjected to controlled alpha-amylase digestion such that a branched glucan having a molecular weight between about 10⁶ D to about 10⁴ D is formed. In another embodiment, gelatinized starch is subjected to controlled alpha-amylase digestion such that linear glucan having a molecular weight between about 10⁵ D to about 10⁴ D is formed.

In an embodiment of the invention, the gelatinized starch is subjected to controlled alpha-amylase digestion such that the average molecular weight of the starch is decreased by at least 10-fold and preferably by at least 100-fold.on digestion.

In an embodiment of the invention, the gelatinized starch is subjected to controlled alpha-amylase digestion such that the average molecular weight of the branched glucans in the treated starch is decreased by at least 10-fold and preferably by at least 100-fold compared to the starch starting material.

In an embodiment of the invention, the gelatinized starch is subjected to controlled alpha-amylase digestion such that the average molecular weight of the linear glucans in the treated starch is decreased by at least 10-fold and up to 100-fold compared to the starch starting material.

In an embodiment of the invention, gelatinized starch is partially digested employing about 1-500 units of alpha-amylase for a time ranging from about 1 minute to about 500 minutes. In another embodiment of the invention, gelatinized starch is digested employing about 1 to about 50 units of alpha-amylase for a time ranging from about 1 minute to about 500 minutes. In a further embodiment of the invention, gelatinized starch is digested employing about 1 to about 50 units of alpha-amylase for a time ranging from about 1 minute to about 250 minutes. In yet another embodiment of the invention, gelatinized starch is digested employing about 10 to about 50 units of alpha-amylase for a time ranging from about 1 minute to about 150 minutes.

In specific embodiments of the invention, the alpha amylase is an animal, fungal or bacterial alpha-amylase. More specifically, the alpha-amylase is pancreatic alpha-amylase or is alpha-amylase obtained from saliva. Mixture of alpha-amylases from different sources may be used. The temperature, pH and other reaction parameters are adjusted for use of a given enzyme.

More specifically, the invention provides a method for making a slowly digestible starch in which a starch is gelatinized. The gelatinized starch is subjected to at least partial retrogradation or crystallization. For example, the gelatinized starch is cooled to a temperature below about 20° C., or more, more preferably to a temperature below about 10° C., but above 0° C. and held at that temperature for a time ranging from about 1 hour to about 48 hours (more preferably for a time ranging froma bout 6 hours to about 24 hours).

The gelatinized partially crystalline native starch is subjected to controlled alpha-amylase digestion to preferentially digest shorter chain length branches of the amylopectin of the native starch. The alpha-amylase treated starch is collected, optionally precipitated with ethanol, optionally washed to remove lower molecular weight-soluble hydrolysis products, and/or optionally dried to provide a slowly digestible starch.

In a specific embodiment, the alpha amylase-treated starch is precipitated from solution by ethanol, cooled to a temperature below about 20° C. (and more preferably to a temperature below about 10° C.), but above 0° C., for a time ranging from about 1 hour to about 24 hours, and collected by centrifugation.

In another specific embodiment, collected starch is washed one or more times with water to remove lower molecular weight water-soluble, alpha-amylase digestion products.

In specific embodiments, the alpha amylase-treated starch is dried, e.g., spray-dried, flash-dried, drum-dried and/or freeze-dried. In another specific embodiment, the moisture level of the alpha-amylase treated starch is adjusted to a desired value. In another specific embodiment, the alpha-amylase treated starch is prepared as a slurry or paste in a selcted amount of water. Such slurries or pastes can be employed in food preparation.

In another embodiment, the gelatinized starch is subjected to one or more steps of temperature cycling to facilitate starch retrogradation prior to treatment with alpha-amylase. For example, gelatinized native starch is subjected to a temperature cycle of cooling to a temperature below about 20° C. (preferably to a temperature below about 10° C.), but above 0° C., for a time ranging from about 1 hour to about 48 hours and thereafter warming the gelatinized starch to a temperature of about 30° C. or more, but less than 100° C., for a time period ranging from about 1 hour to about 48 hours and thereafter cooling to a temperature of below about 20° C. (preferably to a temperature below about 10° C.), but greater than 0° C., for a period of 1 hour to 48 hours. More preferably, the temperature of the gelatinized starch is cycled between about 4° C. (for 1-24 hours, preferably 6-24 hours) and about 30° C. or more (for about 1-24 hours, preferably 6-24 hours).

In another embodiment, the alpha-amylase treated starch is subjected to one or more steps of temperature cycling to facilitate starch retrogradation. For example, alpha-amylase treated starch is subjected to a temperature cycle of cooling to a temperature below about 20° C. (preferably to a temperature below about 10° C.), but above 0° C., for a time ranging from about 1 hour to about 48 hours and thereafter warming the gelatinized starch to a temperature of about 30° C. or more, but less than 100° C., for a time period ranging from about 1 hour to about 48 hours and thereafter cooling to a temperature of below about 20° C. (preferably to a temperature below about 10° C.), but greater than 0° C., for a period of 1 hour to 48 hours. More preferably, the temperature of the alpha-amylase treated starch is cycled between about 4° C. (for 1-24 hours, preferably 6-24 hours) and about 30° C. or more (for about 1-24 hours, preferably 6-24 hours).

In specific embodiments, the starch used as a starting material for digestion is corn starch. In other specific embodiments, the starch starting material contains about 50% or more by weight of amylopectin. In other specific embodiments, the starch starting material contains about 15% to about 35% by weight of amylose. In other specific embodiments, the starch starting material is a waxy starch, particularly a waxy corn starch.

In another specific embodiment, the starch starting material is a starch exhibiting an altered amylopectin structure obtained from a plant mutant or variant, such as wx, alwx, duwx, suwx, aedux corn mutants or analogous mutants having altered amylopectin of other plants. Of particular interest are starches in which the amylopectin is characterized by having a higher proportion of longer chain branches than are typically found in the amylopectin of native starches. See: Obanni, M. and BeMiller, J. N. (1995) Identification of starch from various maize endosperm mutants via ghost structures. Cereal Chemistry 72:436-442 and references therin for description of maize mutants.

The processes of this invention for making slowly digestible starch can be modified by replacing alpha-amylase, in whole or in part, with beta-amylase.

The invention provides slowly digestible starches made by the methods of this invention and food products containing the slowly digestible starches. Of particular interest are food products with lowered glycemic index and/or exhibiting extended energy release after eating. Additionally, the slowly digestible starches of this invention are useful for food products with improved nutritional or dietary benefit and for food products beneficial or therapeutic for diabetic individuals.

### BRIEF DESCRIPTION OF THE DRAWINGS

FIG. 1 is a graph comparing digestion rates of alpha-amylase treated (Sample B, 15 Units alpha-amylase/g starch, 1 hr (squares), Sample C, 30 Units alpha-amylase/g starch, 2 hours(triangles); Sample D, 45 Units alpha-amylase, 4 hr (circles)) and untreated cooked corn starch (diamonds). Equivalent reducing sugar value of maltose is plotted against digestion (with alpha-amylase) time.

FIG. 2 is a graph of glucose response for a rat feeding study (via oral gravage) of untreated cooked corn starch and cooked alpha-amylase treated (Sample C, 30 Units alpha-amylase/g starch, 2 hr) corn starch. Blood glucose concentration (mg/dl) is plotted against time (min) after gavage.

FIGS. 3A-3D are graphs illustrating the results of HPSEC/MALLS assays of alpha-amylase treated starch. The y-axis on the left is molar mass (g/mol), the y-axis on the right is Relative Refractive Index (arrows indicate the y-axis of the different curves). The x-axis is volume (mL) eluting from the HPSEC column. FIG. 3A illustrates the results for cooked, normal corn starch. FIGS. 3B-D illustrates the results for cooked, alpha-amylase treated corn starch with increasing treatment time and increased amount of enzyme as indicated on the graphs.

FIGS. 4A-D are graphs illustrating debranching analysis of untreated and alpha-amylase-treated corn starch. In these figures, absorbance of the eluting material at 490 nm (solid line) measures total carbohydrate (y-axis on left). Absorbance of carbohydrate and iodine complex at 630 nm (dotted line) measures the presence of relatively longer chains of starch (y-axis on left). The figures also plot (y-axis, right) the degree of polymerization (DP) as a function of elution volume.

FIGS. 5A-D present the results of chain-length distribution of debranched samples of native and alpha-amylase treated starch analyzed using a high-performance anion-exchange chromatography equipped with an amyloglucosidase reactor and a pulsed amperometric detector (HPAEC-ENZ-PAD). The results for control (untreated corn starch) are illustrated in FIG. 5A and for alpha-amylase treated starch samples in FIGS. 5B-D.

FIG. 6 compares X-ray diffraction patterns of normal corn starch and alpha-amylase treated samples B, C and D prepared as in Example 1. The alpha-amylase treated samples exhibit crystalline structure. Normal corn starch exhibits an A starch pattern. Alpha-amylase treated starch samples exhibit diffraction patterns different from that of the normal corn starch. The patterns observed can be characterized as more similar to B starch patterns.

FIGS. 7A-7D compare X-ray diffraction patterns of altered digestibility starch before and after cooking. FIG. 7A illustrates that crystallinity of normal corn starch is significantly decreased on cooking. In contrast, in FIGS. 7B-7D, the cooked alpha-amylase treated starch samples exhibit crystallinity.

FIG. 8 is a graph illustrating the results of viscosity measurements as a function of increasing shear rate for starch pastes made using several commercially available resistant starches (Novalose™) and three samples of alpha-amylase treated starch of the present invention.

### DETAILED DESCRIPTION OF THE INVENTION

The invention herein describes processes to make slowly digestible starches. The treated starches of this invention have a range of starch digestion rates and fall between cooked, normal, untreated commercial corn starch, and currently available commercial resistant starches. These slowly digestible starches retain their desirable digestion characteristic after cooking, and accordingly can be used in a range of processed food products. The slowly digestible starches of this invention can be used to modulate the rapid glucose release typical of many processed starchy foods. Target foods for inclusion of a slowly digestible starch ingredient include baked goods, pasta, snack foods, and breakfast foods. Generally, a slowly digestible starch can be employed in food, nutrient or drug preparations. A slowly digestible starch can be used to, at least in part, replace a portion of the starch currently employed in any food, nutrient or drug preparation. More specifically, a slowly digestible starch of this invention can be used, for example, in a liquid or solid processed food, nutritional supplement, or pharmaceutical dosage form (tablet, emulsion, suspension, etc.).

The slowly digestible starch preparations can provide processed foods with lowered glycemic index and which increase satiety. Food products, both solid and liquid, including beverage products, containing slowly digestible starches are generally more healthy foods than processed foods containing similar levels of more rapidly digestible starch. Such food and drink products would be useful for weight management, the treatment of obesity, and for health maintenance and treatment of diabetic and prediabetic individuals. Food and drink products containing slowly digestible starch may also provide health maintenance and treatment benefits for individuals exhibiting glucose intolerance, insulin resistance, and hyperglycemia. Food and drink products containing slowly digestible starch may also provide health benefits by improving cardiovascular indicators.

Slowly digestible starches can also be employed as an extended energy release ingredient, i.e., via extended glucose release, in food products, e.g., snack bars and sports drinks; for use by athletes for performance enhancement and/or to provide for improved concentration and/or memory.

Slowly digestible starches of this invention which retain starch rheologic functionality are of additional use in food, nutrient and drug applications as functional agents to provide desirable rheological properties, for example to provide desired viscosity properties (e.g., thickening); improved consistency, mouthfeel, desirable texture (i.e., organoleptic properties), stickiness, improved emulsion stability, improved flow and like properties, in addition to lowered glycemic index and extended energy release. Slowly digestible starches of this invention can be employed for example as food grade thickening agents, texturizing agents, and stabilizing agents. As indicated by the ability to form starch pastes with the alpha-amylase treated starches, these materials retain at least in part rheologic properties and water absorption or binding capacity of untreated starches which are useful functional properties of starches.

Slowly digestible starches of this invention can be combined with starches exhibiting rapid digestion (e.g., native and/or commercial starches) and/or resistant starches to provide desired digestibility and glucose release and functional properties in food, nutritional and drug applications. Slowly digestible starches can further be combined with other functional ingredients (e.g., agents affecting viscosity or organoleptic properties) such as food grade gums, gelling agents, and the like to modify or adjust rheologic or other properties of a food, drink, nutritional supplement or oral therapeutic or drug preparation.

Starch is composed of two broad classes of polymers, amylose and amylopectin which are assembled to form a starch granule. The lower molecular weight amylose is a mainly linear polymer of alpha 1-4 bonded anhydroglucose units while amylopectin is a branched polymer comprised of linear chains of alpha 1-4 linked anhydroglucose units with branches resulting from alpha 1-6 linkages on the linear chains. Amylose readily reassociates or retrogrades following gelatinization to form less digestible starch material. Commerical resistant starches include those which are highly retrograded amylose. Amylopectin, which is much larger than amylose, is highly branched structure that retrogrades and crystalized much slower and less completely than amylose. Amylopectin is generally highly digestible even after reassocation. This invention is based at least in part on the use of controlled alpha-amylase digestion to change the structure of amylopectin, to make it less branched (but not debranched) and facilitate its reassociation/recrystallization. It was surprizingly found that shorter chains of amylopectin were preferentially digested on treatment with alpha-amylase. The amylopectin of alpha-amylase treated starch was converted into branched glucans (branched 1-4 glucans) having a higher portion of long chains. The invention is also in part based on the partial digestion of at least partially retrograded amylase to form intermediate length linear glucans. (These intermediate length glucans are significantly higher in molecular weight than the highly retrograded amylose of currently available resistant starches.) The branched glucans and intemediate length linear glucan produced by controlled alpha-amylase digestion are believed to provide for slow digestibility.

The controlled digestion of starch with alpha-amylase was found to provide crystalline starch materials. It is believed that the crystallinity of the alpha-amylase treated starches is due to the formaiton of stable crystals from the branched glucans and intermediate length linear glucans resulting from partial digestion. The alpha-amylase treated starch maintains most of its crystalline character after cooking. (In contrast, normal native starches lose their partial crystallinity on cooking.) The alpha-amylase treated starches were found to be more slowly digested than untreated native starch. It is believed that the more crystalline structure of the alpha-amylase treated starch is responsible for its slowly digestble property.

Amylopectin structure with a higher proportion of long chains are believed to be digested more slowly than the amylopectin with a higher proportion of short chains. Branched glucan with higher proportions of longer chain branches are also believed to be digested more slowly. Additionally, retograded intermediate length amylose is likewise believed to be digested slowly, and in particular, more slowly than retrograded native chain amylsoe.

The slowly digestible starches of the invention include alpha-amylase treated starches having low dextrose equivalent (D.E.). .D.E. is the reducing power of a starch hydrolysate expressed as a per cent of the reducing power of the same weight of D-glucose. The higher the D.E., the lower the average molecular weight of the product. The maximum D.E. is 100, which equivalent to dextrose. Slowly digestible starches of this invention include those which have D.E. of about 2 or less and particularly those having D.E. of 1 or less. D.E. is measured by methods well-known in the art., for example the Fehling Volumetric Titration method can be used.

The slowly digestible starches of this invention have low glycemic index which can be determined by methods well-known in the art (Wolever, T., Jenkins, D. J. A., Jenkins, A. L., and Josse, R. G. (1991) The glycemic index: methodology and clinical implications. American Journal of Clinical Nutrition 54:846-854.). The slowly digestible starches of this invention can be employed to make edible products, including liquid and solid food products, which exhibit lower glycemic index compared to analogous products containing rapidly digested starch.

The molecular weight and the extent of branching of alpha-amylase treated starches can be assessed employing art-known methods, particularly size-exclusion high performance chromatography (HPSEC) coupled with a light scattering detector (MALLS). Such methods can be used to analyze the distribution of chain lengths in branches and the degree of polymerization.

The unqualified term starch is used herein to refer to starch that is generally suitable for use in the methods of this invention to make slowly digestible starch. Starch includes all starches as they are extracted from any and all plant sources. Native starches include all starches as found in nature in any plant source. Starches can also be obtained from plants which are obtained by standard plant breeding methods as well as by mutagenesis and genetic engineering or by combination of mutagenesis and genetic engineering with standard plant breeding methods. Plant sources for starches include cereals, legumes, tubers, roots and fruits. Starch can be extracted from corn (maize), rice, barley, wheat, oat, sorghum, oat, pea, sago, tapioca (cassava), arrowroot, sweet potato, yams, and banana, for example. Starches can be extracted from various mutant plants which exhibit alterations in starch phenotype.

Starch also includes commercial starches that may be washed, bleached or otherwise treated to remove undesired components.

Native starch from different types of plants generally may contain different percentages of amylose and amylopectin, different size starch granules and different polymeric weights for amylose and amylopectin. As a result, native starch from different plant sources may have significantly different properties. Typically, the, amylose content of starches ranges from about 15% to about 35%. Waxy starches contain higher levels of amylopectin (90% by weight or more) and are extracted from plants such as waxy maize, waxy rice, waxy barley and waxy sorghum. High amylose starches contain greater than about 50% by weight amylose. High amylose starch can be subdivided into starches containing between about 50 to 60%, 70 to 80% by weight amylose and very high amylose starches which have 95% or more by weight amylose. In general, all such native and non-native starches, and mixtures thereof, are useful as starting materials in the methods herein. However, it is generally preferred that the starch starting material contain 40% or more by weight of amylopectin. Waxy starches are a particularly interesting starting material for the methods of this invention.

Resistant starches are not useful starting materials for the processes herein. A resistant starch is a starch or starch derivative which is not digestible in the small intestine. Officially the term is reserved for the sum of starch and products of starch degradation not absorbed in the small intestine of healthy individuals. Resistant starches can be physically inaccessible starch (RS1, e.g. trapped in seeds), granular starch (R2), highly retrograded starch (R3) and chemically modified starch (R4). The slowly digesting starches of this invention are substantialy digested (at least about 80%) in the human small intestine. They are more slowly digested than native or commercial starches from which they are derived, as exemplified by normal corn starch, but are more rapidly digested compared to resistant starches, such as highly retrograded starches.

In general starches can be characterized by structural and functional properties. Starches can be characterized by their amylose/amylopectin (or branched glucan) content; by molecular weight measurements (e.g., peak molecular weight, component molecular weight, average molecular weight, etc. dependent upon methods employed), by branching analysis, by X-ray diffraction measurement, by differential scanning calorimetry (DSC), by chromatography, digestion analysis (in vitro or in vivo), by determination of dextrose equivalents, assessment of glycemic index by viscosity and other rheological properties, by water absorption or binding capacity, among a number of other well-known properties. Characterization of such properties can be assessed as a function of different appropriate variables such as time, types of cooking (e.g., high and low moisture cooking), application of stress, moisture level, etc.) Such characterization can be made using methods that are well-known and in some cases standard in the art. All such methods of analysis and characterization of starch that are known in the art or functional equivalents of such methods can be employed to assess the structural and functional characteristics of starches made by the methods herein to compare and contrast those starches to untreated starches, native starches, commerical starches and/or resistant starches.

Parbal digestion with alpha-amylase as employed in the methods of this invention is believed to preferentially remove shorter branches from amylopectin and to provide intermediate length linear glucans. Starches which contain amylopectin with altered structures or in which the ratio of amylose to amylopectin is altered are particularly useful as starting materials for the processes herein to make slowly-digestible starches. Single, double and triple mutants, for example, are known in the art which exhibit such altered starch and/or amylopectin phenotypes. For example, mutant starches from waxy, aewx, duwx, suwx, and aeduwx mutants of corn, rice, barley, sorghum and other plants containing amylopectin fractions with different fine structures are useful herein to produce slowly digestible starch.

In the method of this invention for production of slowly digestible starch, gelatinized starch is subjected to controlled partial digestion (or hydrolysis) employing alpha-amylase. The digestion is controlled by employing specified amounts of alpha-amylase, preferably dilute alpha-amylase, for specified digestion times. Additionally, the pH and or the temperature of the reaction can be controlled, dependent upon the alpha-amylase employed, to control digestion. Digestion is controlled to obtain a product of desired molecular weight or containing a component of desired molecular weight, or to obtain a product exhibiting a desired branching pattern. Alpha-amylase treated starch can be assessed by methods well-known in the art and as described in the examples herein by X-ray diffraction, chromatography, molecular weight measurement, debranching analysis and digestibility analysis to adjust digestion conditions to achieve slowly digesting starches having functional and structural properties as described in herein and as exemplified in the slowly digestible starches specifically exemplified herein.

It will be appreciated by those of ordinary skill in the art that the partial digestion of a given gelatinized starch to achieve a desired digested product can be standardized by monitoring the amount of hydrolysis. For example, hydrolysis can be monitored by measuring the concentration of reducing groups freed by enzyme digestion to define a digestion end point that provides a digestion product with desired properties. Alternatively, changes in molecular weight (as noted elsewhere herein) or changes in physical or chemical properties of the starch can be used to define an end point for partial hydrolysis. Digestion can then be controlled by monitoring a given variable or property to detect the desired endpoint when digestion should be stopped.

Starches are digested by addition of a selected amount of a selected alpha-amylase to an aqueous slurry containing up to about 50% solids, but more typically 5% to about 20% solids. The pH and temperature of the digestion mixture are adjusted to obtain a desired level of hydrolysis and are generally dependent upon the enzyme employed. Most generally, the pH of the digestion reaction can range from about 3.0 to about 8.0, but is dependent upon the specific enzyme employed. Most generally, the temperature of the digestion reaction can range from about 20° C. to about 50° C., but again is dependent upon the enzyme used. Heat-stable alpha-amylases may be employed at generally high temperatures, ranging up to about 90° C. The amount of enzyme employed and the digestion time can be adjusted if necessary dependent upon the amount of starch present in view of the descriptions herein and the specific examples herein to achieve a desired extent of hydrolysis. The digestion reaction may be stirred or agitated by any known method that does not disrupt the digestion reaction. As is known in the art, enzyme reactions can also be carried out employing immobilized enzymes (immobilized on beads, columns or like substrates) in contact with the enzyme substrate. Immobilized alpha-amylase can be employed in the digestion method herein.

The digestion time can be controlled by selective deactivation of the enzyme by any method, e.g., boiling, autoclaving, or change in pH.

In exemplified embodiments, alpha-amylase is used to partially digest gelatinized starch. Alpha-amylase (also called 1,4-alpha-Dglucan glucanohydrolase) catalyzes the endohydrolysis of 1,4-alpha-glucosidic linkages in oligosaccharides and polysaccharides, including starch. Alpha-amylase acts on starch, glycogen and related polysaccharides and oligosaccharides in a random manner, liberated reducing groups in the alpha-configuration. Various alpha-amylases are known in the art and can be obtained from animal, plant, bacterial and fungal sources. Alpha-amylases from porcine pancreas, human saliva, barley malt, Aspergillus oryzae, and Bacillus species are commercially available (Sigma). In addition, heat-stable alpha-amylases are also commercially available (e.g., from Bacillus licheniformis.) A unit of alpha-amylase will liberate 1 mg of maltose from starch in 3 min. at pH 6.9 at 20° C. The use of alpha-amylase from porcine pancrease is specifically exemplified herein. Those of ordinary skill in the art, in view of the descriptions and examples herein, can readily employ alpha-amylases from other sources for use in the methods herein.

Starches are gelatinized prior to partial digestion with alpha-amylase. Generally, any method for gelatinizing starch can be employed. Gelatinization is an irreversible process in which starch granules in water swell and are disrupted. The temperature at which a starch gelatinizes is the gelatinization temperature and, as is known in the art, it depends upon the type of starch used. Starch can be gelatinized by heating in water solutions or by pressurized heating of water solutions, as can be achieved by autoclaving. The temperature and time of heating and or autoclaving to achieve gelatinization can vary with the type of starch employed. Additional methods known in the art for gelatinizing starch are exemplified in U.S. Pat. Nos. 5,149, 799, 5,131,953, 5,037,929, and 4,465,702. It may be beneficial to combining heating with stirring and autoclaving methods to obtain uniform gelatinization of starch samples. Typically, the starch samples to be gelatinized are about 5% to about 15% by weight starch in water (or buffered aqueous solution.) It will be apparent tho those of ordinary skill in the art that pregelatinized starches can be employed in the methods herein. Pregelatinized starches would be hydrated an appropriate level, allowed to retrograd, if necessary or desirable, and subjected to controlled alpha-amylase digestion.

Gelatinized starches are optionally, but preferably, at least partially retrograded or crystallized prior to enzymatic digestion. Partial retrogradation or crystallization can be achieved by methods known in the art. In particular, gelatinized starch can be at least partially retrograded or crystallized by cooling to a temperature below about 20° C. (without freezing) and holding the gelatinized starch at that temperature for a time ranging from about 1 hour to about 48 hours. More typically, at least partial retrogradation is achieved by cooling the gelatinized starch to a temperature below about 10° C. (without freezing) for a period of 6 to about 24 hours. Often in small batch preparations, the gelatinized starches are cooled overnight. Most typically, the gelatinized starch is cooled to about 4° C. The specific cooling temperature and cooling time can depend upon the type of starch starting material used, the water content of the gelatinized starch, the amount of gelatinized starch being processed and the equipment being used.

Optionally, one or more temperature cycling steps can be applied to the gelatinized starch before controlled enzymatic digestion. In such temperature cycling steps, the temperature of the gelatinized starch is cycled between a selected high and a selected two temperature with the gelatinized starch being held at the low temperature for a selected time and at the high temperature for a selected time. The selected times can be different and most generally range from about 1 hour to 48 hours and more typically from about 6 hours to about 24 hours. An often practical time for holding the gelatinized starch at a given temperature is overnight. The low cycling temperature is below about 20° C. (without freezing), and more preferably below about 10° C. without freezing. The high cycling temperature is greater than about 30° C. (without boiling). The specific high and low temperatures and holding times can depend upon the type of starch starting material use, the water content of the gelatinized starch, the amount of gelatinized starch being processed and the equipment being used. Temperature cycling can be applied as many times as is desired or need to achieve a desired result, but will typically applied at least once and repeated if desired until no change in crystalline properties are observable or until a desired crystalline pattern is achieved. The amount of water in the gelatinized starch sample may be adjusted to a desired level during any cooling and/or temperature cycling.

Optional, cooling and or temperature cycling steps analogous to those applied to the gelatinized starch prior to digestion can be applied to the controlled-digestion, alpha-amylase treated starch before it is precipitated, collected and/or dried. In a specific embodiment, the method of making slowly digestible starch comprises at least one step of cooling of the alpha-amylase treated starch to a temperature below about 20° C., or more preferably below about 10° C. (without freezing) and holding the alpha-amylase treated starch at that temperature for a time ranging from 1 hour to about 48 hours (more preferably about 6 hours to about 24 hours). In another, specific embodiment, the method comprises applying to the alpha-amylase treated starch at least one temperature cycling step between a low temperature of less than about 20° C., or more preferably below about 10° C. (without freezing) and a high temperature of greater than about 30° C. with holding times at those temperatures ranging from about 1 to about 48 hours.

The alpha-amylase treated, optionally temperature processed starch is collected and the moisture content in the collected starch is adjusted to a desired level. The collected starch will typically be dried, although starches with varying amounts of water or moisture content, including starch water slurries, can be employed in the preparation of products. Any means known in the art to reduce or adjust the water content of a starch can be employed and any means known in the art can be used to dry the starch. Flash-drying, freeze-rying and spray-drying can all be employed. Each of these drying methods is well-known in the art and can be readily applied to drying the starch product of the methods herein. In a preferred embodiment, the starch is spray-dried. The type of drying method that is applied can depend upon the amount of material to be dried, the amount of water present, and a balance between the effectiveness, speed and cost of the drying method.

The starch product of the methods herein can be collected by precipitation and centrifugation or filtration. Precipitation can be facilitated by addition of ethanol to a water/starch mixture. Ethanol is employed in particular for food grade applications, but other precipitation agents may be employed. The amount of ethanol added is adjusted to range up to about 25% by volume ethanol but is more typically about 5% to about 10% by volume. After addition of ethanol, the mixture is allowed to stand with cooling to achieve precipitation. The time that the mixture is allowed to stand may be adjusted to maximize precipitation, but is typically at least about 1 hour to about 24 hours. A practical time for small batch processing is overnight. After addition of ethanol, the mixture may optionally be cooled to a temperature below about 20° C. (without freezing) for a selected holding time (about 1-48 hours, but more typically about 6 hours to about 24 hours).

Collected starch and dried collected starch can be subjected to modification normally applied to starches by any physical treatment or reagent that does not effect the desired digestiblity properties and food grade quality of the starch. For example, the dried collected starch can be subjected to low moisture heating processes, as such are known in the art.

The processes of this invention for making slowly digestible starch can be modified by replacing alpha-amylase, in whole or in part, with beta-amylase. Beta-amylase (also called beta-1,4-D-glucan maltohydrolase, saccharogen amylase, or glycogenase) catalyzes hydrolysis of 1,4-alpha-glucosidic linkages in polysaccharides, including starch, to remove successive maltose units from the non-reducing ends of the chains. Various beta-amylases are known in the art from plant and bacterial sources. For example, beta-amylase (Type I-B) from sweet potato and beta-amylase (Type II-B) are available from commercial sources (Sigma.) A unit of beta-amylase activity is defined as the amount of enzyme that will liberate 1.0 mg of maltose from starch in 3 min. at pH 4.8 at 20° C. In generally, beta-amylase is employed as described herein for alpha-amylase. Those of ordinary skill in the art can readily adjust the specific digestion conditions (e.g., pH, temperature), amount of enzyme and digestion time in view of the descriptions and examples provided herein for use of alpha-amylase. Beta-amylase treated staches will exhibit slow digestibility properties similar to those observed with alpha-amylase treated starches and will be useful in food, nutritional and drug applications as described herein for alpha-amylase treated starches.

Digestibility of starch can be assessed by invivo and invitro methods that are known in the art. The method exemplified herein in the exampels is an in vitro method which is believed to provide a good measure of the relative digestibility of different starh samples. Simulated digestion by the method of Englyst et al., European Journal of Clinical Nutrition (1992) 46: S33-S50 can also be employed. ThThe various in vivo and invitro methods for assessing digestibility are discussed in Vonk et al. (2000) A, J. Clinical Nutr. 72:432-438.

The slowly digestible starches of this invention can be employed as ingredients in food and beverage products, in edible solid and liquid products, in liquid and solid nutritional supplements and in edible liquid or solid drug preparations. The amount of slowly digestible starch that is added to a food, nutritional or drug product is selected to achieve desired functional properties (rheological, organoleptic, or like properties) digestibility rates and energy or glucose release rates or a desirable balance of those properties. Food products or nutritional or therapeutic preparations of this inventon can generally can comprise between about 0.01% to about 100% by weight of a slowly digestible starch. More typically edible products comprise between about 1% to about 50% by weight of slowly digestible starch.

Slowly digestible starches of this invention can replace all, or only a portion of, normal digestiblity starch in an edible product. Slowly digestible starches of this invention can, for example, replace 50% or less of normal digestiblity starch in an edible product. Slowly digestible starches of this invention can replace all or only a portion of resistant starch in an edible product. Slowly digestible starches of this invention can, for example, replace 50% or less of resistant starch in an edible product.

The slowly digestible starches of this invention can be employed as an ingredient in baked goods (cakes, cookies, pastries and the like), pasta, snack bars, cereals (ready-to-eat or cereals intended to be cooked), confectionary, dressings, fillings, icing, sauces, syrups, gravies, puddings, custards, processed dairy compositions (e.g., processed cheese, youghurts and creams), soups, beverages, sports drinks, and sustained energy release foods and snacks, such as energy bars.

The slowly digestible starches of this invention can be used to prepare edible products exhibiting lower glycemic index than similar products prepared with untreated more rapidly digestible starches.

Slowly digestible starches of this invention can retain at least some of the functional properties, e.g., rheological properties, exhibited by more rapidly digestible starches (e.g., native starches) which may be lost in resistant starches. For example, slowly digestible starches of this invention can be employed to form stable pastes (e.g., stable for 12 or more hours). Thus, slowly digestible starches can be employed as functional food grade additives which provide benefical rheological properties (viscosity, mouth feel, texture, consistency (organoleptic properties) emulsion or suspension stability, water-absorption or binding capacity or flow properties to a food or other edible product. More specifically, slowly digestible starches can be employed as food grade thickening agents and texturizing or texture-modifying agents.

### THE EXAMPLES

### Example 1

### Preparation Of Slowly-Digesting Starch

Normal corn starch (˜25% amylose, 5% by weight in water) was heated to over 80° C. with stirring to disperse the starch and begin gelatinization and thereafter gelatinized in an autoclave (at ˜120° C.) for 15 minutes. The gelatinized starch solution is cooled to and stored at 4° C. for 12 hours to allow the starch to retrograde. The starch solution is then warmed to 37° C., and partially digested using alpha-amylase [porcine pancreas alpha-amylase (Sigma A-3176) containing 15.4 units/mg of solid at pH=6.9; a unit being defined as the amount of enzyme that will liberate 1.0 mg of maltose from starch in 3 min at pH 6.9 at 20° C.; added at 15 units/g starch, 30 units/g starch, or 45 units/g starch]. Partial digestion was accomplished by treating the starch solution (with pH adjusted to 6.9, starch solution was made up to 0.0625 mM sodium glycerophosphate-HCl, 1.5625 mM NaCl and 0.3125 mM CaCl₂) for 1-4 hours with alpha-amylase. Enzyme action was stopped at a selected time by boiling or autoclaving (for 15 min).

Ethanol (10% of the original volume of the starch solution) was added to the alpha-amylase treated starch solution to facilitate precipitation, the mixture was cooled to 4° C. and stored overnight. The starch preparation was then collected by centrifugation at 6000×g for 15 min. The collected starch was then washed with half of the original volume of water and collected by centrifugation twice. The collected residual starch was mixed with half the original volume of water and spray-dried to obtain slowly digesting starch product (atomizer temperature 120° C., drying temperature 75° C.). Digestibility can be assessed by an in vitro or in vivo starch digestion assay. In general, digestibility of the starch product decreases with increasing enzyme digestion times.

### Example 2

### In Vitro Procedure Of Testing Cooked Starch Digestion

Starch (500 mg) was cooked in 5 mL distilled water for 10 minutes and cooled to 37° C. Buffer (20 mL, 1 mM sodium glycerophosphate-HCL, pH 6.9, 25 mM NaCl, 5 mM CaCl₂) was then added to the cooked starch. The solution was equilibrated at 37° C., and 150 Units of alpha-amylase were added [0.5 mL, 12.3 units/mg of porcine pancreas alpha-amylase (Sigma A-3176) containing 15.4 units/mg of solid at pH=6.9; one unit will liberate 1.0 mg of maltose from starch in 3 min at pH 6.9 at 20° C.). Enzyme hydrolysis was carried out at 37° C. and 0.5 mL aliquots of hydrolyzed solutions were withdrawn at selected times. The equivalent reducing sugar value of maltose was determined using the Nelson-Somogyi method for determination of reducing sugars (Chaplin, M. F., and Kennedy, J. F. (1994) Carbohydrate Analysis—A practical Approach. 2^{nd} Edition. pp 4. Oxford University Press Inc., Oxford, UK).

The extent of hydrolysis was determined by the amount of reducing sugars produced by hydrolysis and digestion curves were produced. Appropriate untreated starch controls were performed.

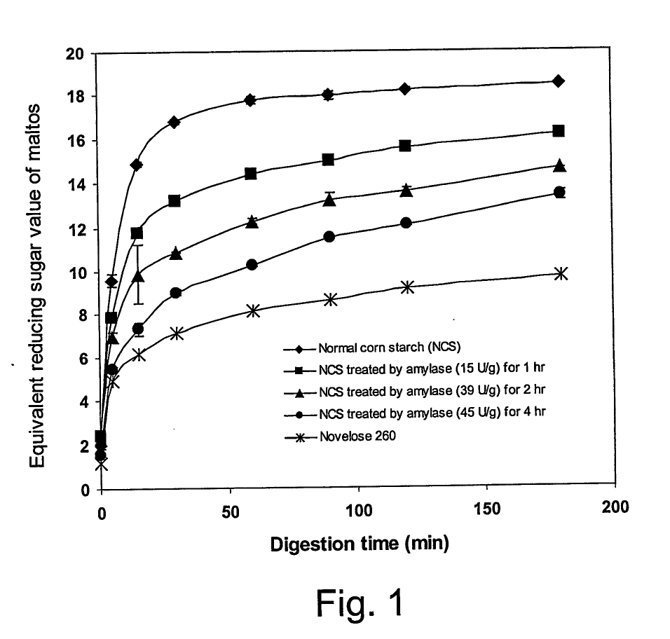

FIG. 1 is a graph of digestion rates of spray-dried alpha-amylase treated normal starch (corn starch) products compared to digestion of normal gelatinized (cooked) corn starch as evaluated by measurement of equivalent reducing sugar. Digestion rates of the spray-dried alpha-amylase treated starches were reduced by 20% to about 50% (percentage decrease from normal corn starch at 30 min and 180 min (3 hr) digestion).

### Example 3

### In Vivo Assay Of Acute Glycemic Response To Cooked Treated Starch

Starch samples (10% by weight in water) were cooked in boiling water for 10 min and cooled at room temperature for 1 hour. Sprague/Dawley rats (age of 44-48 days and about 175-199 gram) were fed 2.3 mL (10% water starch solution) of a cooked starch sample and blood samples were drawn at selected times after feeding. Profiles are of blood glucose levels over 180 min after ingestion of starches (by oral gravage). Blood glucose levels were determined by the calorimetric method, using a Cobas Mira Plus autoanalyzer (Roche Diagnostics, Mannheim, German.) (See: A.Gokcel, H. Karakose, E. M. Ertorer, N. Tanaci, N. B. Tutuncu and N. Guvener (2001) “Effects of Sibutramine in Obese Female Subjects With Type 2 Diabetes and Poor Blood Glucose Control” Diabetes Care 24:1957-1960.)

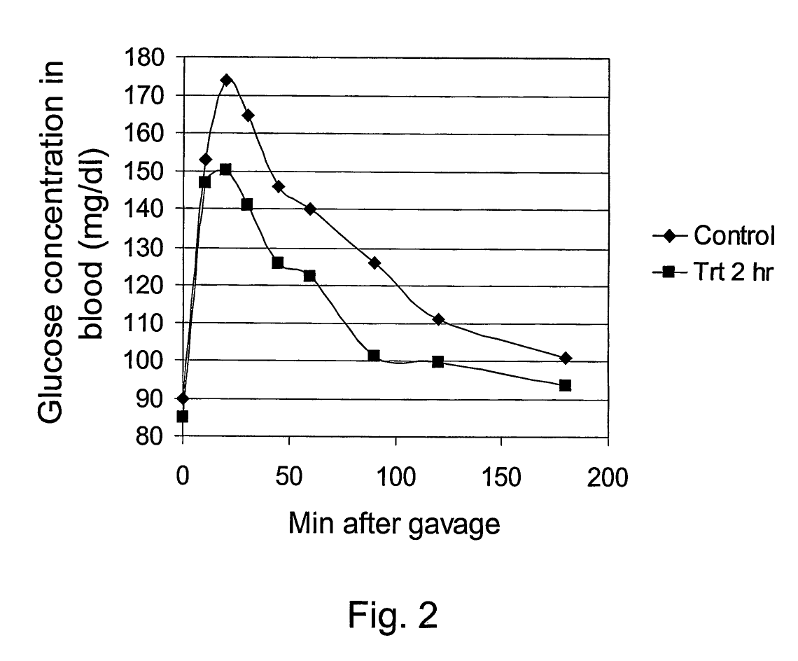

FIG. 2 is a graph comparing glucose concentration as a function of time after oral gravage feeding of normal cooked starch and a sample of alpha-amylase treated starch (Sample C, 30 Units enzyme for 2 h). The results indicate that there is a signficant decrease in glycemic index (area under the curve) of the amylase-treated starches of this invention compared to that of normal cooked corn starch. See Wolever, T., Jenkins, D. J. A., Jenkins, A. L., and Josse, R.G. (1991) The glycemic index: methodology and clinical implications. American Journal of Clinical Nutrition 54:846-854 for methods for measurement of glycemic index.

### Example 4

### Molecular Weight Profiles Of Treated Starch

Molecular weight profiles of spray-dried, alpha-amylase-treated slowly digesting starch compositions were assessed using high performance size-exclusion chromatography (HPSEC) coupled with a multiangle laser-light scattering (MALLS) and a refractive index (RI) detector. (See: S. G. You and S.-T. Lim (2000) “Molecular Characterization of Corn Starch Using an Aqueous HPSEC-MALLS-RI System Under Various Dissolution and Analytical Conditions” Cereal Chem. 77(3):303-308; and S. G. You and S.-T. Lim (1999) “Molecular Characterization of Wheat Amylopectins by Multiple Laser Light Scattering Analysis,” Cereal Chem. 76(1):116-121.

Starch samples (100 mg) were dissolved in 95% DMSO (10 mL) and boiled with continuous stirring (stir bar) for 1 hr. The samples were cooled to room temperature, and stirred for additional 24 hr. Solubilised samples were precipitated with ethanol (40 mL), and centrifuged at 3800 g for 15 min. The precipitates were washed with 40 mL ethanol and collected with centrifugation at 3800 g for 15 min for two times. The precipitates were dried for 24 h in vacuum at room temperature. Dried samples (15 mg) were added with 5 mL purified water and boiled with stirring for 10 min. The samples were cooked in a pressure cooker (about 121° C.) for 25 min, and were immediately injected into a HR 16/50 Pharmacia column (Sephacryl S500HR gel, exclusion range Mr 4×10⁵-2×10⁷, Pharmacia, Sweden), that was connected to a Varian 9012 HPLC solvent delivery system with 1 mL loading loop. The detectors were Varian 9040 Reflective Index Detector (Varian Associates, Inc. Walnut Creek, Calif.) and a multi-angle laser light scattering (Wyatt/Optilab 903 Interferometric Refractometer and DAWN DSP Laser Photometer, Wyatt Technology Corporation, Santa Barbara, Calif.). Mobile phase was a 0.02% aqueous solution of sodium azide filtered through a 0.22 μm GV Durapore membrane (GA Durapore Membrane Filters, Millipore, Ireland). The flow rate was 1.5 mL/min.

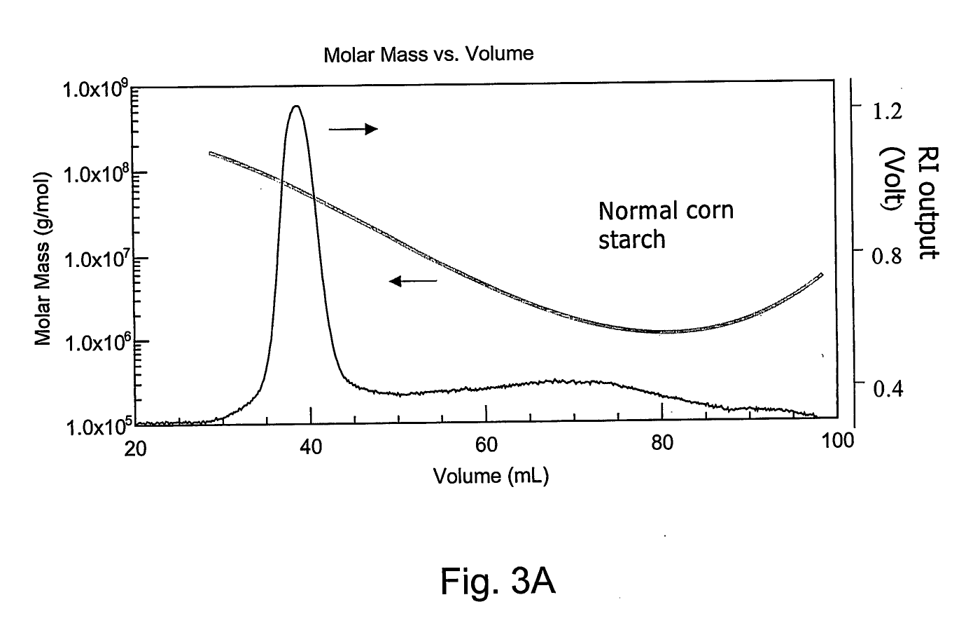

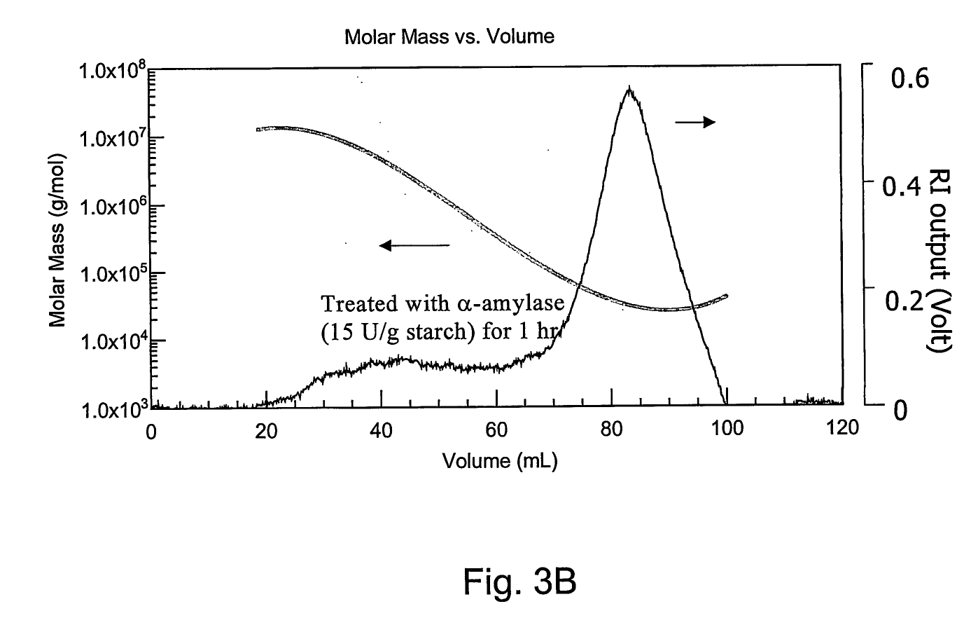

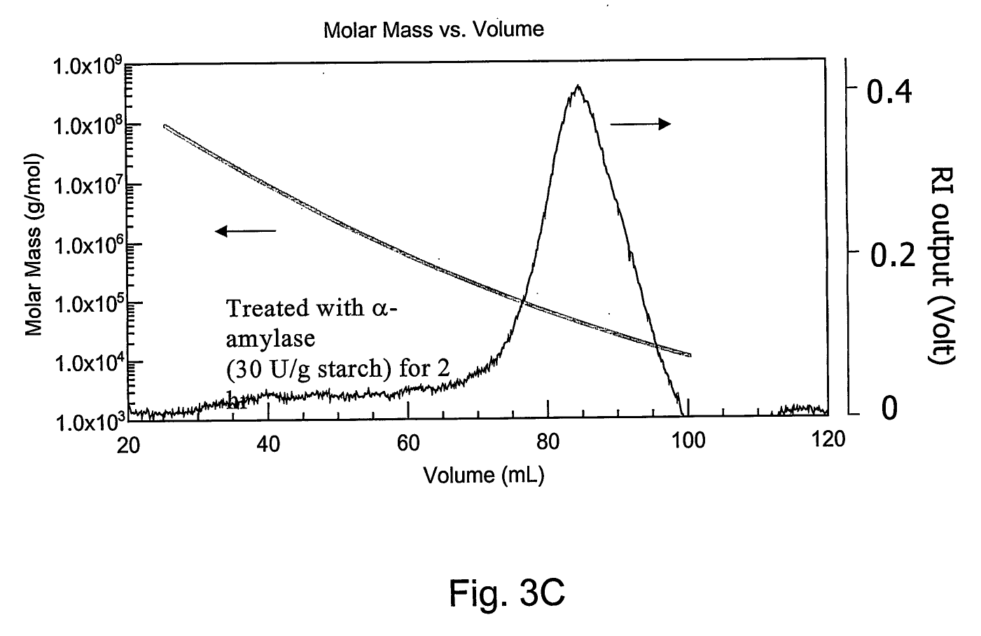

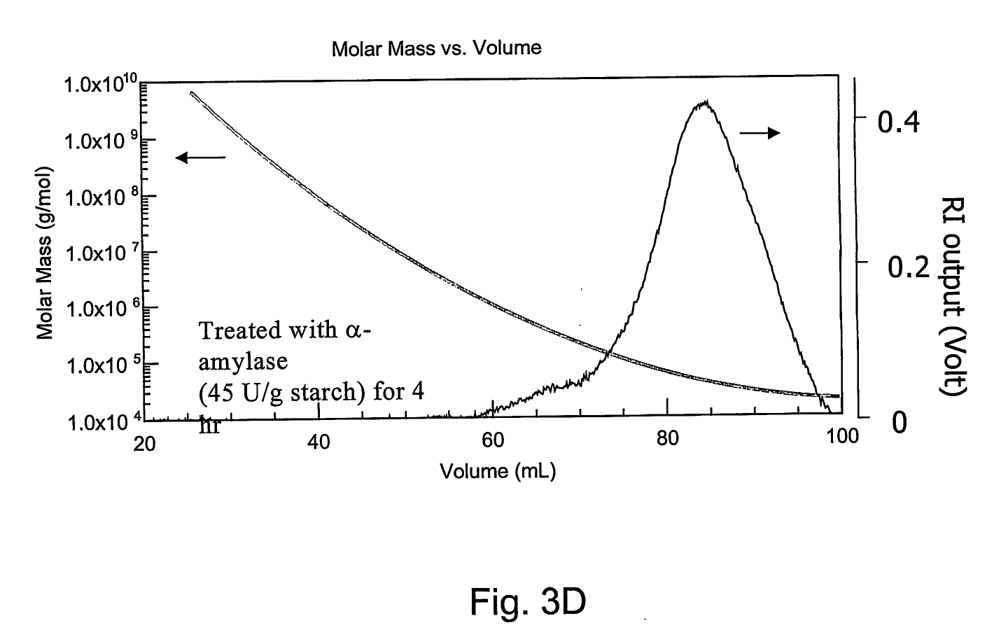

Molecular weight profiles obtained by the HPSEC/MALLS technique are illustrated in FIGS. 3A-3D. FIG. 3A shows the chromatograph and molar mass distribution of a control normal corn starch. FIG. 3B is the chromatograph and molar mass distribution of alpha-amylase treated corn starch (Sample B, treated with 15 Units enzyme/g starch, 1 h). FIG. 3C illustrates results for alpha-amylase treated corn starch (Sample C, treated with 30 Units enzyme/g starch, 2 h.) The molecular weight of the amylopectin peak of the control starch sample was measured to be close 1×10⁸ D (FIG. 3A). With alpha-amylase treatment, the molecular weight of the amylopectin fraction decreases indicating digestion of the amlyopectin and reduction of its molecular weight (FIGS. 3B-C). With higher alpha-amylase concentration and digestion time (FIG. 3D) amylopectin is substantially digested to lower molecular weight materials.

### Example 4

### Fine Structure Analysis Of Alpha-Amlyase Treated Starch Compositions

Fine structure analysis was performed by treating starch compositions with isoamylase to debranch amylopectin into linear chains and the examining the linear chains that resulted from debranching (Han, X.-Z., and Hamaker B. R. Amylopectin fine structure and rice starch paste breakdown. 2001. Journal of Cereal Science 34 (3): 279-284.) The analysis was performed to determine what portion(s) of the amylopectin molecule were more highly digested during partial alpha-amylase treatment and to obtain information of the structure of the product that resulted from digestion. Starch samples (100 mg) were dissolved in 95% DMSO (10 mL) and boiled with continuous stirring (stir bar) for 1 hr. The samples were cooled to room temperature, and stirred for additional 24 hr. Solubilised samples were precipitated with ethanol (40 mL), and centrifuged at 3800 g for 15 min. The precipitates were washed with 40 mL ethanol and collected with centrifugation at 3800 g for 15 min for two times. The precipitates were dried for 24 h in vacuum at room temperature. Dried starch samples (6 mg) were dissolved in 1 ml purified water by heating in a boiling water bath for 20 min. After cooling to room temperature, 1.5 ml acetate buffer (0.1 M, pH 4.0) and 10 μl isoamylase (Megazyme, Ireland) were added to the solution. The suspension was incubated in a 40° C. water bath for 24 hr. The enzyme-substrate reaction was stopped by heating the solution in a boiling water bath for 10 min. The suspensions were fractionated by descending chromatography on a Bio-gel P-10 (exclusion range Mᵣ 1500-20,000, Bio-Rad Laboratories, Hercules, Calif.) column (1.6×53 cm) operating at a flow rate of 0.2 ml/min using water containing 0.02% sodium azide as eluant. Fractions (3 ml) were collected. Aliquots of the fractions (0.5 ml) were analyzed for total carbohydrate using the phenol-sulfuric acid method. Fractions were also analyzed by iodine binding, and the iodine-polysaccharide complex was scanned for its absorption maximum (λₘₐₓ) used to determine the average degree of polymerization (DPₙ). The following equation provided by Fales (1980) (Fales, F. W. The linear relationship between iodine staining and average chain length of the unbranched amyloglucans. Biopolymers 19 (1980) 1535-1537) was used to calculate the DPₙ: DPₙ=3290/(635-λₘₐₓnm). Debranching procedures and measurements were performed at least in duplicate.

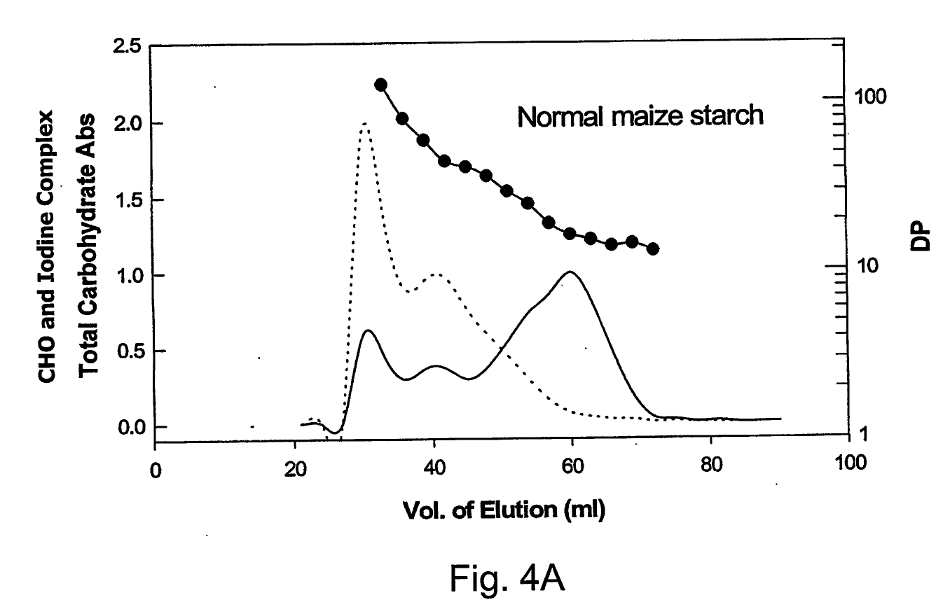

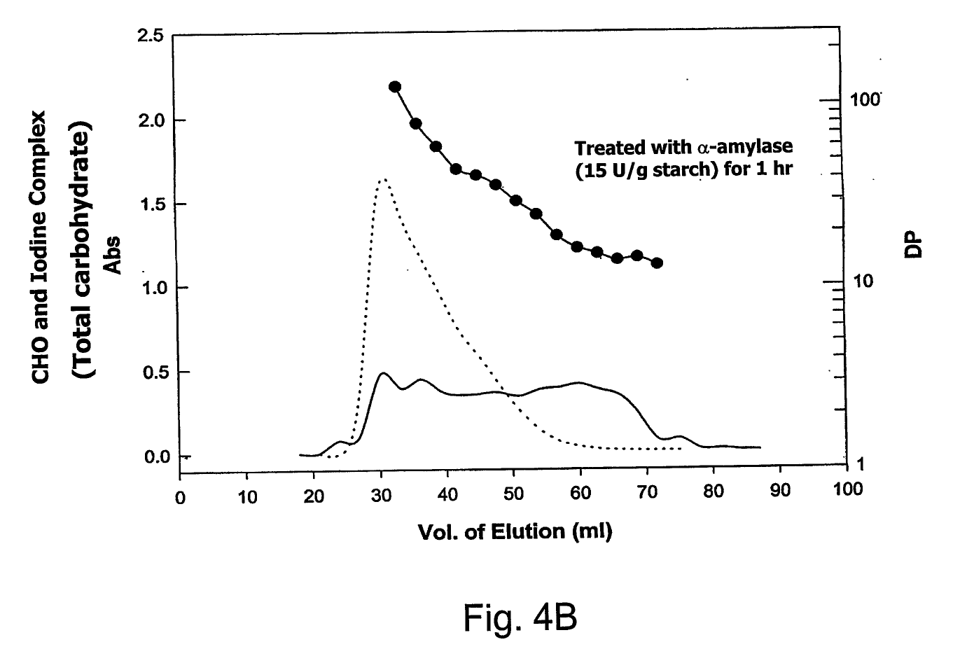

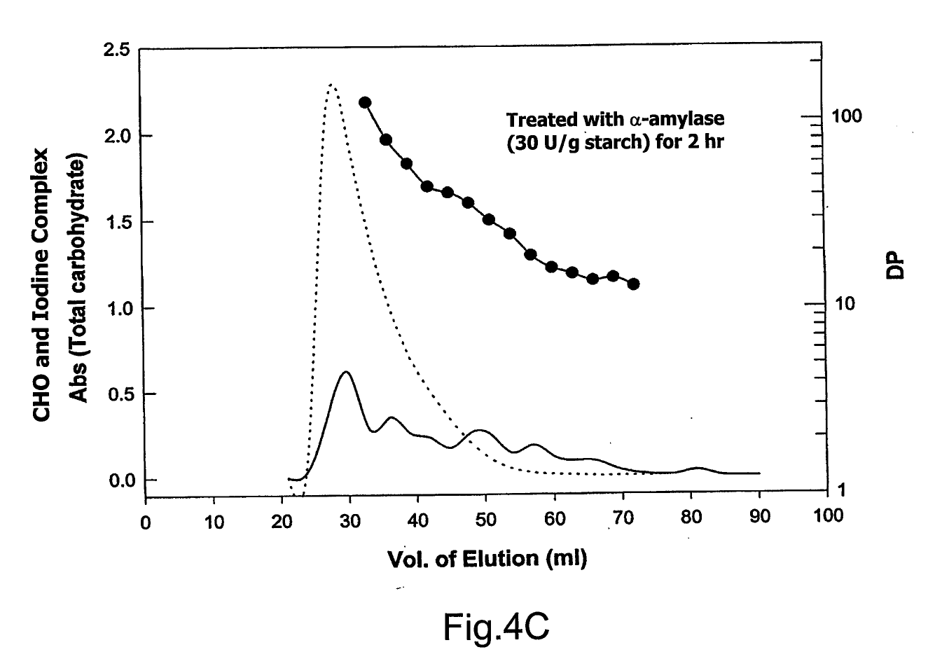

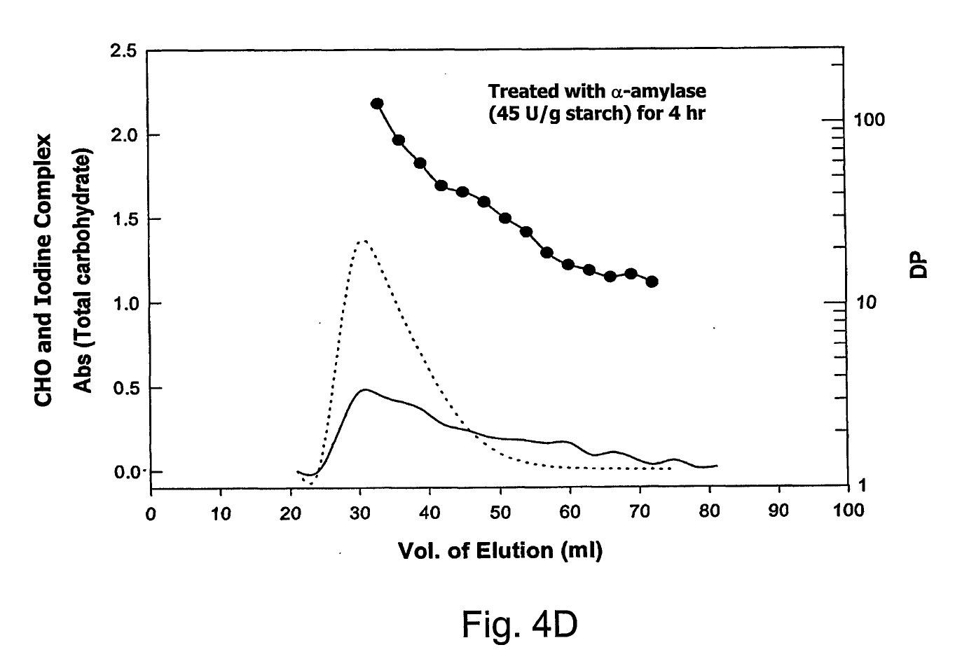

FIGS. 4A-D are graphs illustrating debranching analysis of untreated and alpha-amylase-treated corn starch. In these figures, absorbance of the eluting material at 490 nm (solid line) measures total carbohydrate (y-axis on left). Absorbance of carbohydrate and iodine complex at 630 nm (dotted line) measures the presence of relatively longer chains of starch (y-axis on left). The Figures also plot (y-axis, right) degree of polymerization (DP) as a function of elution volume.

The results illustrated in FIGS. 4A-4D indicate that a high proportion of short linear chains of starch were digested quickly by alpha-amylase and that intermediate and long chains of starch were digested relatively slowly. The intermediate and long chains of starch appear to provide the slowly-digesting property of the treated starches. Very long chains of starch (DP about 100 or above) remaining after partial alpha-amylase digestion, likely from amylose, appear to form a more resistant starch.

Additional analysis of disbranched starch samples was conducted. Debranching of starch samples was done following the procedure of Jane, J., & Chen, J. F. (1992) “Effect of amylose molecular size and amylopectin branch chain length on paste properties of starch” Cereal Chemistry,69, 60-65. Chain-length distribution of debranched samples were analyzed by using a high-performance anion-exchange chromatography equipped with an amyloglucosidase reactor and a pulsed amperometric detector (HPAEC-ENZ-PAD) (Dionex, Sunnyvale, Calif.) following the procedures reported by Wong, K. S., and Jane, J. (1997). Quantitative analysis of debranched amylopectin by HPAEC-PAD with a post-column enzyme reactor. Journal of Liquid Chromatograph, 20, 297-310

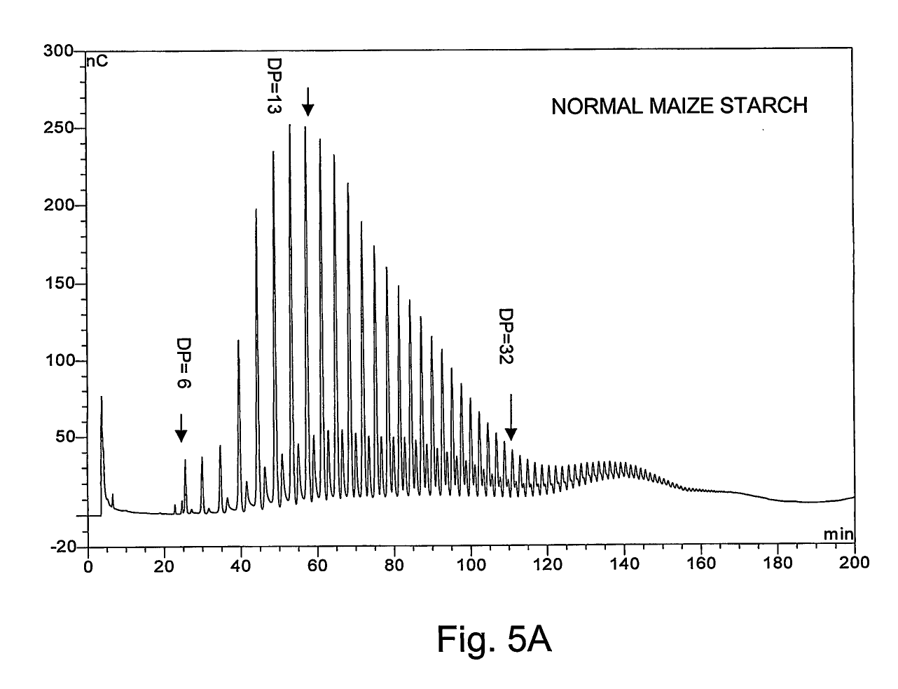

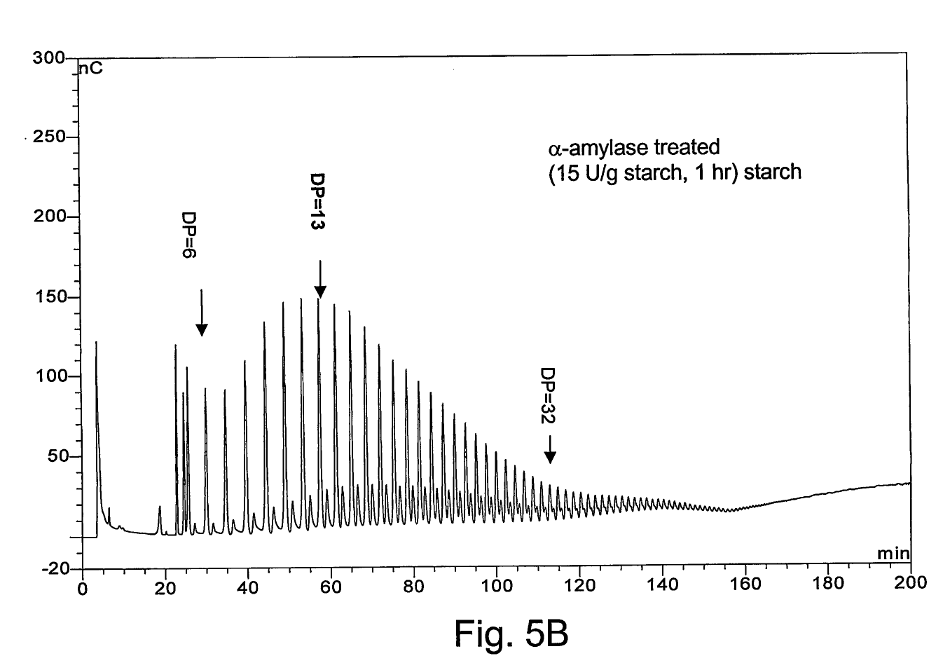

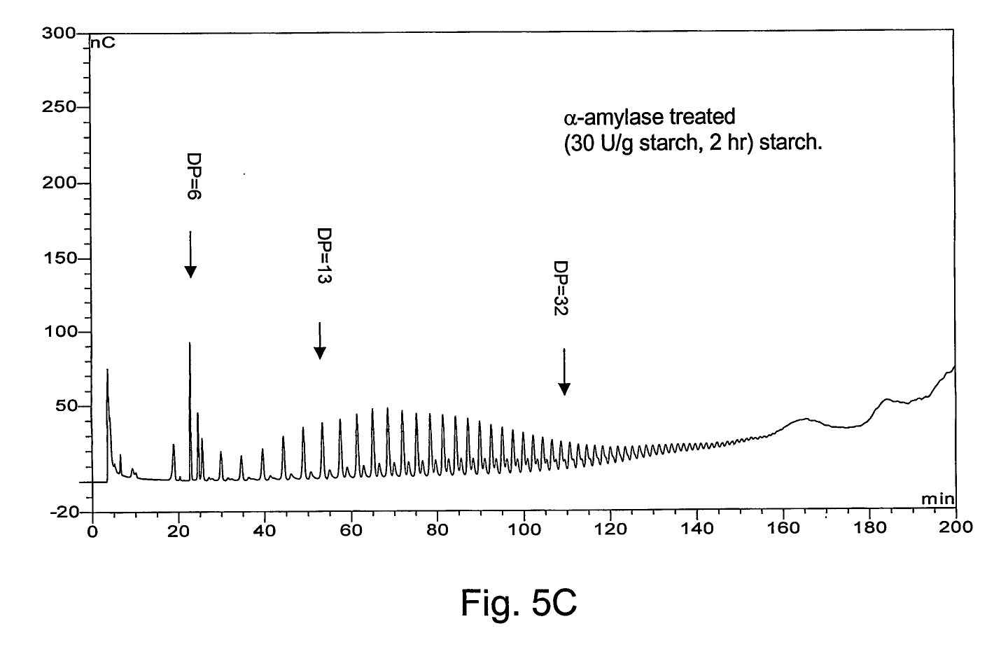

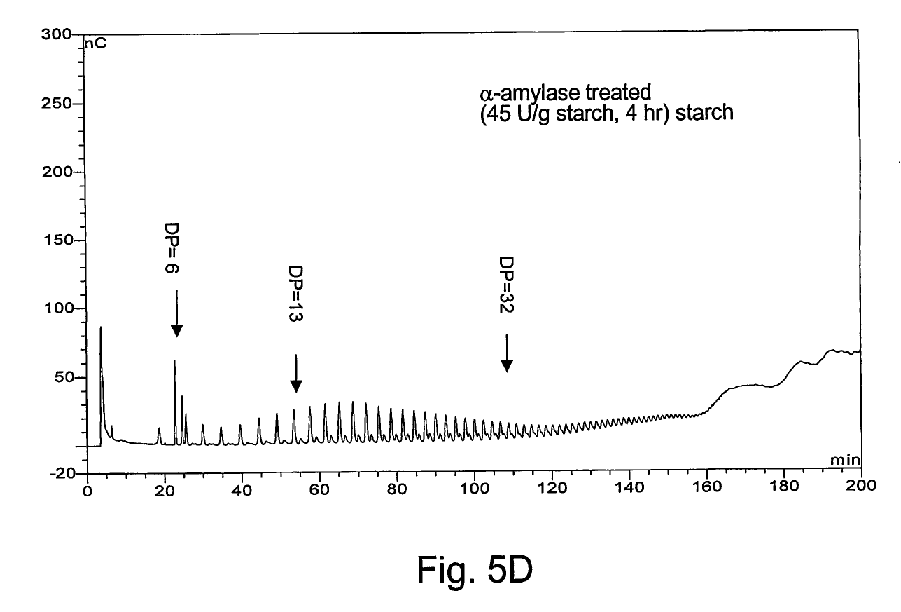

The separation of a sample with the system employed a PA-100 anion-exchange analytical column and a guard column (Dionex, Sunnyvale, Calif., USA) with a gradient composed of eluent A (100 mM sodium hydroxide) and eluent B (100 mM sodium hydroxide, 300 mM sodium nitrate). The separation gradient was: 0-5 min, 99% A and 1% B; 5-30 min, linear gradient to 8% B; 30-150 min, linear gradient to 30% B; 150-200 min, linear gradient to 45% B. Each sample was analyzed in duplicate. The results for control (untreated corn starch) are illustrated in FIG. 5A and for alpha-amylase treated starch samples in FIGS. 5B-D. This method provides the distribution of short chains that results from debranching. The results confirm that the alpha-amylase-treated starch (Samples B-D) retain a branched structure. In addition, the results show that the released branches having a degree of polymerization centered on about peak 13 (DP 13) in the control untreated corn starch are significantly decreased on alpha-amylase treatment. The alpha-amylase-treated starches which on average contain longer branch chains than native starch are believed to have reduced digestibility.

One method for providing a quantitative comparison of how the branching profile is changing as a function of alpha-amylase treatment is to compare the height (or area) ratios of two or more selected peaks representing branches of different length. For example the relative ratio of peaks at DP13 and DP32, indicated on the figures, in normal untreated corn starch is about 10/1. As the extent of hydrolysis of the starch is increased (Sample B-D), the ratio of the 13/32 peaks decreases to about 6/1 (Sample B) and about 3/1 (Samples C and D). This change in peak ratio increases that the branching distribution is shifting to higher DP as the extent of digestion increases. In this case, the change in peak ratio can be used as a measure of the extent of hydrolysis by the alpha-amylase. The gelatinized starch is treated with alpha-amylase to obtain a starch product that exhibits a DP13/DP32 ratio that is less than about 10, between about 74 and about 3. This same type of branching peak anaylasis can be done with other pairs of peaks in the data collected.

### Example 5

### X-Ray Characterization of Alpha-Amylase Treated Starches

The alpha-amylase treated (see, Example 1) and untreated normal corn starch samples were assessed by X-ray diffraction. Moisture level of normal corn starch and spray-dried alpha-amylase-treated normal corn starches was equilibrated in desiccators containing a saturated solution of K₂CO₃ at room temperature for at least a week. In addition, the effect of cooking on the crystalline properties of the starches was assessed. Starches (10% in water) were cooked in boiling water for 10 min and immediately transferred to a 37° C. water bath. After 30 min, the starch solutions were frozen (−20° C.) overnight. The starch samples were freeze-dried (VirTis Genesis 25 ES, VirTis, Gardiner N.Y.). Moisture level of freeze-dried samples was also equilibrated in a desiccator containing saturated solution of K₂CO₃ at room temperature for at least a week. About 1 g of the powdered starches was packed into an aluminum holder and x-ray data were collected at room temperature on a Philips PW3710 diffractometer (Philips, USA) with a step width of 0.01° in the 2° range 10-35°. CuKα(λ=1.5418 Å) radiation was used and the tube was operated at 40 kV and 25 mA. The time spent at each step was 3 s. The diffraction patterns were obtained from normal corn starch, spray-dried alpha-amylase-treated normal corn starches, and their cooked and freeze-dried forms.

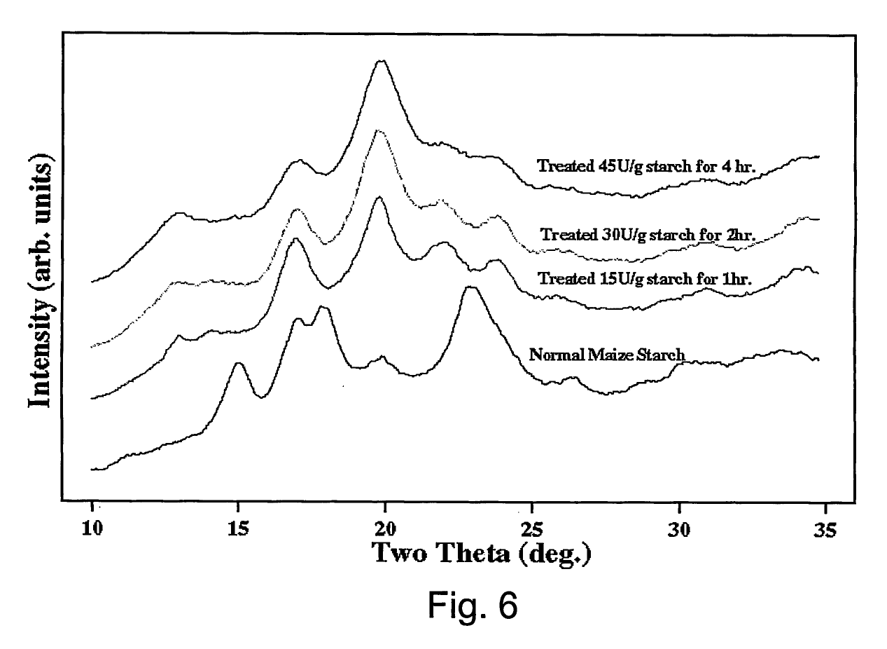

The comparative results are illustrated in FIG. 6 for uncooked normal corn starch and alpha-amylase treated samples B, C and D. The X-ray diffraction patterns show that all of the alpha-amylase treated samples have crystalline structure. The X-ray pattern of the treated starches indicates a transformation in crystal form from A-type in the normal corn starch to more like a B-type structure in the alpha-amylase treated starch, with an increase in the extent of alpha-amylase treatment.

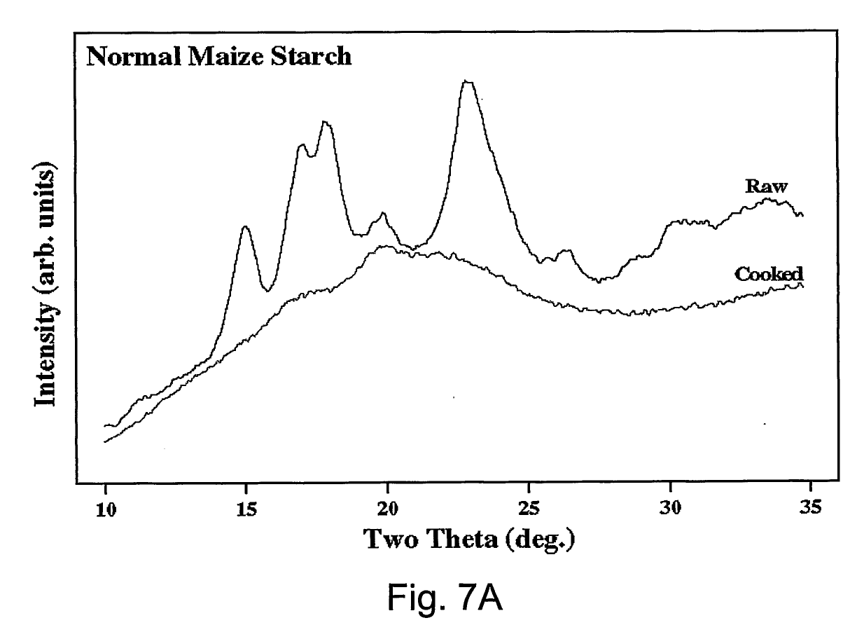

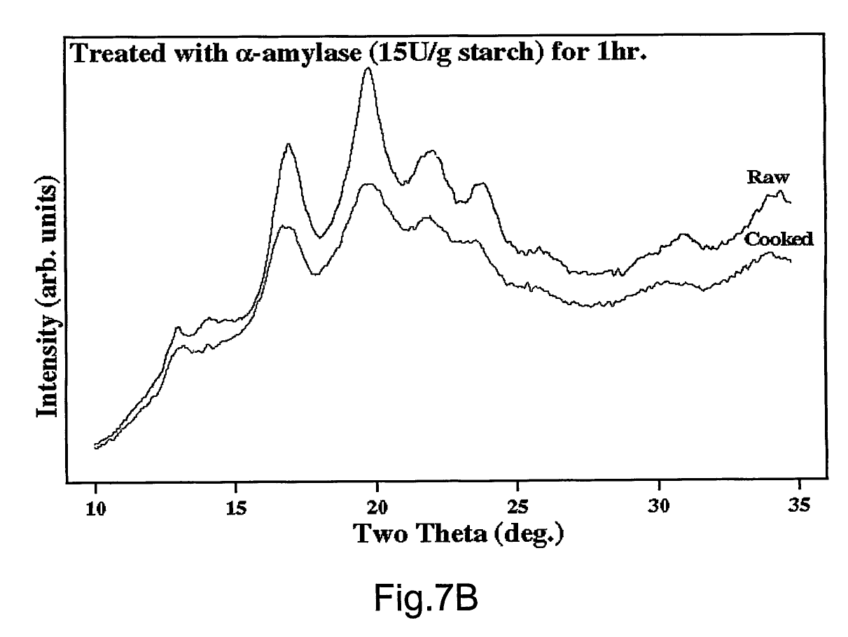

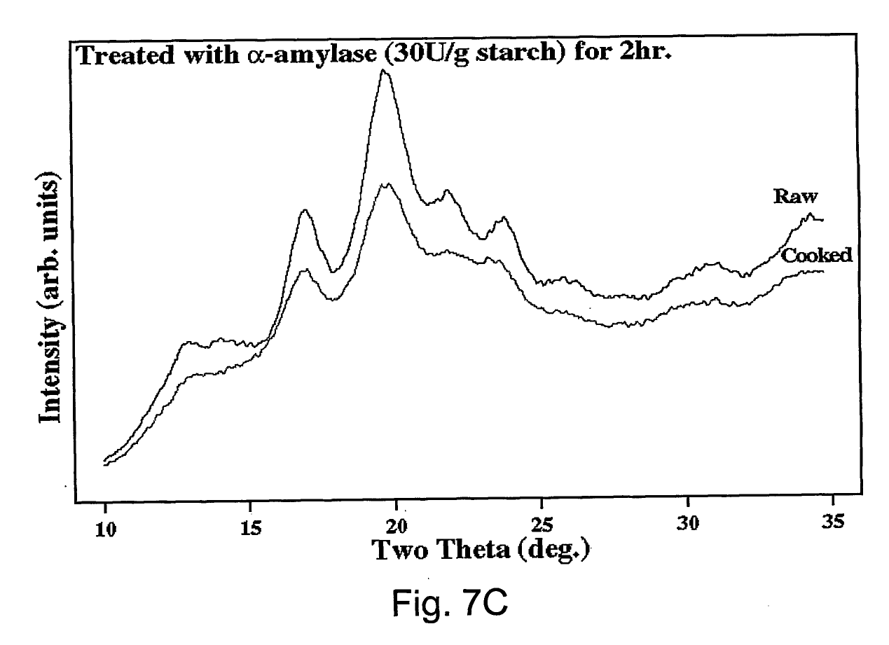

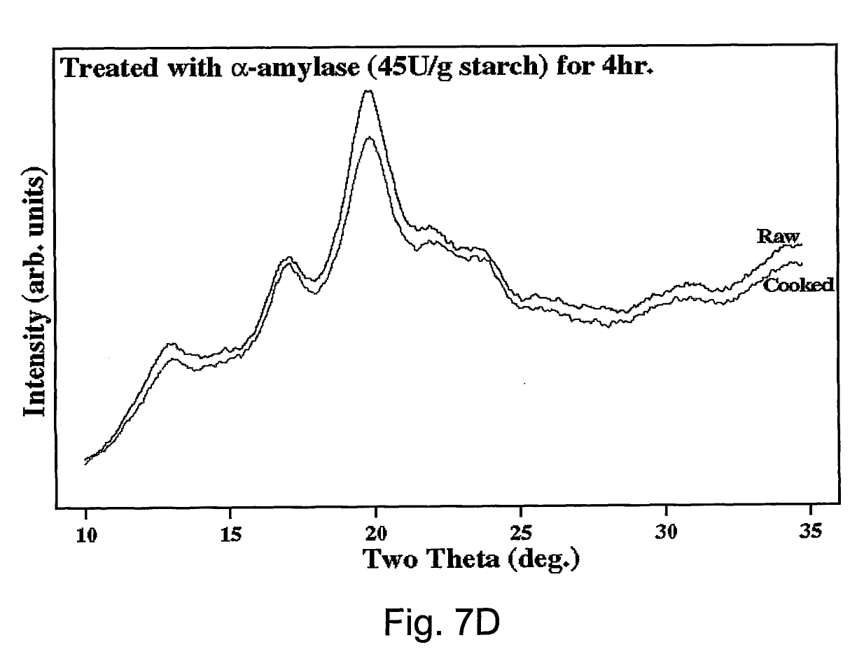

FIGS. 7A-7D compare X-ray diffraction patterns of altered digestibility starch before and after cooking. FIG. 7A illustrates that crystallinity of normal corn starch is significantly decreased on cooking. In contrast, in FIGS. 7B-7D, crystallinity of the alpha-amylase treated starch is significantly retained on cooking. The stable crystal structure in these alpha-amylase treated starches is believed to be responsible at least in part for their slow-digesting characteristics.

### Example 6

### Viscosity Of Alpha-Amylase Treated Starch Solutions

Novelose (resistant starches(as indicated in the Figures), normal corn starch and spray-dried alpha-amylase-treated normal corn starch solutions (10% by weight in water) were cooked in boiling water for 10 min. After cooking, the starch solution was immediately placed in a 27° C. water bath for 2 hr. The starch solution (1.2 ml) was transferred onto the center of the plate of a controlled stress rheometer (ReoLogica Instruments AB, Sweden). Measurements were conducted using a cone and plate system with a cone of 4 cm diameter and 4° angle. Steady shear measurements were conducted at 27° C. using a range of shear rates of 5.6-238 1/s and the resulting flow curves were analyzed. Measurements were done at least in duplicate.

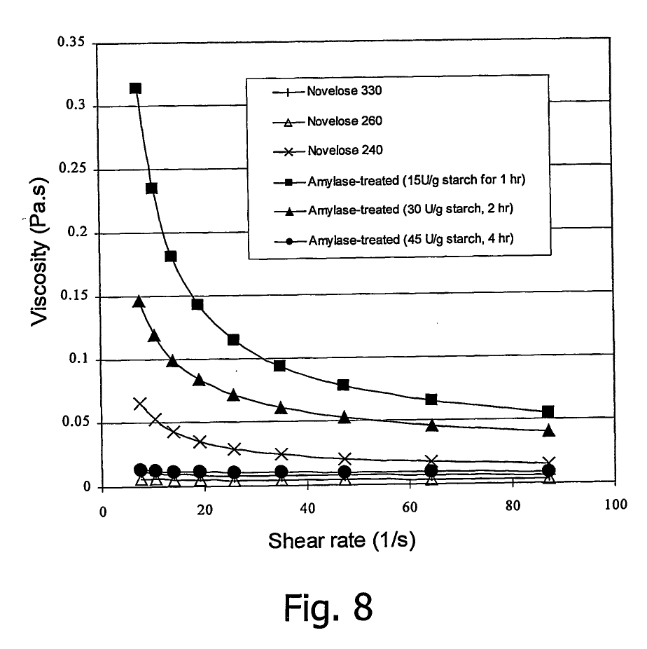

FIG. 8 is a graph illustrating the results of viscosity measurements as a function of increasing shear rate for several commercially available resistant starches (Novalose(( )) and three samples of alpha-amylase treated starch of the present invention. Treated Starches B and C exhibit significantly higher viscosity compared to these commercial resistant starch products. These results indicate that the alpha-amylase treated slow-digesting starches of this invention exhibit desirable rheological properties that are useful in food processing and other applications.

Those of ordinary skill in the art will appreciate that starting materials, reagents, techniques and assay methods other than those specifically noted herein, which are known to those in the art, can be applied to the practice of this invention without resort to undue experimentation. For example, native starches other than those specifically mentioned can be employed. Digestion assays other than those specifically mentioned in the specification can be applied to the characterization of starch materials and products herein. Wherever numerical ranges (e,g., for temperatue, time, weight percentage, etc.) all subranges of the specific ranges given are intended to be encompassed.

1. A method for making a slowly digestible starch which comprises the steps of:
a. gelatinizing a starch which contains amylopectin;
b. cooling the gelatinized starch to a temperature below about 20° C. to allow the gelatinized starch to at least partially crystallize;
c. treating the at least partially crystallized starch with an alpha-amylase to preferentially remove short chain branches from the amylopectin therein; and
d. collecting the alpha-amylase-treated starch to obtain a slowly digestible starch.

2. The method of claim 1 further comprising the step of removing water-soluble hydrolysis products from the alpha-amylase-treated starch.

3. The method of claim 1 wherein the starch is gelatinized by heating a mixture of the starch in water.

4. The method of claim 1 wherein the starch is gelatinized by autoclaving a mixture of the native starch in water.

5. The method of claim 1 wherein the gelatinized starch is cooled to a temperature between about 2020 C. and about 0° C. for a period of about 1-48 hours before digestion with alpha-amylase.

6. The method of claim 1 wherein the gelatinized starch is cooled to a temperature of 4° C. for a period of 6-24 hours.

7. The method of claim 1 further comprising a step of temperature cycling the gelatinized starch by warming the cooled gelatinized starch to a temperature between above about 30° C. for a period of about 1-48 hours.

8. The method of claim 7 further comprising one or more additional temperature cycling steps in which the temperature of the gelatinized starch is cycled between a temperature ranging from about 20° C. to about 0° C. and a temperature ranging from about 30° C. to about 100° C.

9. The method of claim 1 wherein the starch is treated with alpha-amylase such that a substantial portion greater than about 25% of the short A chains of the amylopectin of the starch are removed.

10. The method of claim 1 wherein the gelatinized starch is treated with alpha-amylase under conditions that result in an alpha-amylase treated starch composition in which the branched glucan therein contains a higher amount of longer chain branches than the amylopectin of the starch.

11. The method of claim 1 wherein the gelatinized starch is treated with alpha-amylase under conditions such that the peak molecular weight of the branched glucans of the alpha-amylase treated starch is reduced by at least 10-fold compared to the starch.

12. The method of claim 1 wherein the gelatinized starch is treated with alpha-amylase under conditions such that the peak molecular weight of the branched glucans of the alpha-amylase treated starch is reduced by at least 100-fold compared to the starch.

13. The method of claim 1 wherein the gelatinized starch is treated with alpha-amylase under conditions such that the molecular weight of the branched glucan of the alpha-amylase treated starch is between about 10⁶ D and 10⁴ D.

14. The method of claim 1 wherein the gelatinized starch is treated with alpha-amylase under conditions such that average molecular weight of the alpha-amylase treated starch is between about 10⁶ D and 10⁴ D.

15. The method of claim 1 wherein the gelatinized starch is treated with about 1 to about 500 Units of alpha-amylase for a period of time ranging from about 1-to about 500 minutes.

16. The method of claim 1 wherein the gelatinized starch is treated with from about 10 to about 100 Units of alpha-amylase for a period of time ranging from about 1 to about 300 minutes.

17. The method of claim 1 wherein the gelatinized starch is treated with from about 10 to about 50 Units of alpha-amylase for a period of time ranging from about 10 to about 250 minutes.

18. The method of claim 1 wherein the gelatinized starch is treated with from about 10 to about 40 Units of alpha-amylase for a period of time ranging from about 10 to about 150 minutes.

19. The method of claim 1 further comprising a step of drying the collected alpha-amylase treated starch.

20. The method of claim 1 wherein the collected alpha-amylase treated starch is spray-dried.

21. The method of claim 24 wherein the collected alpha-amylase treated starch is flash dried.

22. The method of claim 1 wherein the alpha amylase treated starch is precipitated by addition of ethanol and collected.

23. The method of claim 1 wherein ethanol is added to a water/alpha amylase treated starch mixture and the ethanol, water, starch mixture is cooled to a temperature between about 20° C. and 0° C. for a time ranging from about 1 to about 48 hours.

24. The method of claim 1 wherein the collected alpha-amylase treated starch is heated to a temperature above 30° C. for a period ranging from about 1-48 h.

25. The method of claim 1 further comprising a step of applying a heat-moisture treatment to the collected alpha-amylase treated starch.

26. The method of claim 1 further comprising a step of annealing the collected alpha-amylase treated starch.

27. The method of claim 1 wherein the starch contains 50% or more by weight amylopectin.

28. The method of claim 1 wherein the starch is a waxy starch.

29. The method of claim 1 wherein the starch contains from about 15% to about 35% by weight amylose.

30. The method of claim 1 wherein the starch is obtained from a grain, tuber or legume.

31. The method of claim 1 wherein the starch is obtained from corn, wheat, rice, barley or sorghum.

32. (canceled)

33. (canceled)

34. The method of claim 1 wherein the starch is extracted from a mutant plant which exhibits an altered starch, amylose or amylopectin phenotype.

35. The method of claim 1 wherein the starch is extracted from a mutant plant designated as a waxy, aewx, duwx, suwx, or aeduwx mutant.

36. The method of claim 35 wherein the mutant starch is a corn, rice, barley, or sorghum mutant.

37. The method of claim 1 wherein the starch is a waxy starch.

38. The method of claim 1 wherein the alpha-amylase is of animal origin.

39. The method of claim 1 wherein the alpha-amylase is a bacterial or fungal alpha-amylase or a mixture thereof.

40. A slowly digestible starch made by the method of claim 1.

41. A food product containing a slowly digestible starch of claim 40.

42. A nutritional supplement or drug preparation containing a slowly digestible starch of claim 40

43. A food product, nutritional supplement or drug preparation which comprises between about 0.1% to about 50% by weight of a starch of claim 40.

44. The food product of claim 41 which is a baked good, pasta, a snack bar, a cereal or a confectionary.

45. The food product of claim 41which is a dressing, filling, icing, sauce, syrup, gravy, pudding, custard, or a soup.

46. The food product of claim 41 which is a sports drink, or a sustained energy release bar.

47. A low glycemic index food product containing a slowly digestible starch of claim 41.

48. A functional food additive comprising a slowly digestible starch of claim 41 present in a functional amount for affecting the viscosity, mouth feel, texture, emulsion or suspension stability, or flow properties of a food product.

49. A thickening agent which comprises a starch of claim 41.

50. The thickening agent of claim 49 which is a food-grade thickening agent.

51. A slowly-digestible starch which comprises branched glucan having a different branching structure than the amylopectin of the starch from which the slowly digestible starch was prepared.

52. The slowly-digestible starch of claim 51 which comprises branched glucan in which the branches released by enzymatic debranching exhibit a molecular weight range higher than the molecular weight range of branches released by enzymatic debranching of the amylopectin from the native starch from which the slowly digestible starch was prepared.

53. The slowly-digestible starch of claim 51 having an average molecular weight between about 10⁷ to 10⁴ D.

54. The slowly digestible starch of claim 53 which has an average molecular weight between about 10⁶ to about 10⁴ D.

55. The slowly digestible starch of claim 53 which has a DE (dextrose equivalent) of 2 or less.

56. The slowly digestible starch of claim 53 which has a DE of 1 or less.

57. (canceled)

58. A slowly digestible starch of claim 40 which comprises less than about 25% amylose.

59. A slowly digestible starch of claim 40 which comprises 25% or more amylopectin.

60. A slowly digestible starch of claim 40 which comprises 50% or more amylopectin.

61. A slowly digestible starch of claim 40 which has a DE of about 2 or less.

62. A slowly digestible starch of claim 40 which has DE of about 1 or less.

63. A food product containing a slowly digestible starch of claim 51.

64. A food product which comprises between about 0.1% to about 50% by weight of a starch of claim 51.

65. A functional food additive comprising a slowly digestible starch of present in a functional amount for affecting the viscosity, mouth feel, texture, emulsion or suspension stability, or flow properties of a food product.
